(19) United States
(12) Patent Application Publication
(10) Pub. No.: US 2024/0165289 A1
(43) Pub. Date: May 23, 2024

(54) FRAGRANCE MACHINE

(71) Applicant: GUANGDONG YENIU TECHNOLOGY CO., LTD., DONGGUAN (CN)

(72) Inventor: JUN LONG, NANCHONG (CN)

(21) Appl. No.: 18/085,726

(22) Filed: Dec. 21, 2022

(30) Foreign Application Priority Data
Nov. 17, 2022 (CN) 202223055392.0

(51) Int. Cl. A61L 9/12

(52) U.S. Cl. CPC A61L 9/12

(57) ABSTRACT

The present invention discloses a fragrance machine, including a housing, an atomizing assembly and a power supply, wherein an opened end of a connecting pipe is communicated with the atmosphere outside the housing; a liquid storage bottle is limited in a hollow part of the housing, and has an opened end detachably fixed with a closed end of the connecting pipe; the closed end of the connecting pipe is provided with connecting holes and a blind mounting hole; an atomizing pipe is embedded in the blind mounting hole; a mounting block provided with a liquid spray hole is fixed at one end of the atomizing pipe; the liquid spray hole is communicated with the interior of the liquid storage bottle; an air nozzle is arranged in the atomizing pipe; the air nozzle is communicated with an air pump; and the power supply is electrically connected with the air pump.

### CROSS REFERENCE TO RELATED APPLICATION

This patent application claims the benefit and priority of Chinese Patent Application No. 202223055392.0 filed on Nov. 17, 2022, the disclosure of which is incorporated by reference herein in its entirety as part of the present application.

### TECHNICAL FIELD

The present invention relates to the field of atomization devices, and more particularly to a fragrance machine.

### BACKGROUND

The fragrance machine atomizes fragrance liquid inside a liquid storage bottle in an atomizing mode, and then discharges the atomized liquid into the air. Some fragrance machines use atomizing pieces, while others use a heating mode. Although the atomization effect of the atomizing mode of the atomizing pieces is good, the atomizing pieces have short service life and are easily damaged; and the heating mode has relatively high power consumption. Besides the above two modes, a method of compression atomization is further used, namely, the compressed air flows through a small nozzle to form high-speed airflow and generate a negative pressure for driving the liquid or other fluids to spray a barrier together, so that the liquid or other fluids splash around under high-speed impact, and the droplets become atomized particles to be sprayed out from an air outlet pipe. This mode has good atomization effect, long service life and low energy consumption. However, the existing compression atomization type fragrance machine has unreasonable atomization structure, and its atomization effect is inferior to the expected effect. Moreover, liquid storage bottles in fragrance machines need to be disassembled and replaced frequently, so most of the liquid storage bottles of the existing fragrance machines on the market are designed inside housings, and are not easy to disassemble and replace. In addition, users cannot intuitively observe the amount of fragrance liquid in the liquid storage bottles during use.

Therefore, how to provide a fragrance machine capable of solving the above problems is an urgent problem for those skilled in the art.

### SUMMARY

In view of this, the present invention provides a fragrance machine.

To achieve the above purpose, the following technical solution is adopted in the present invention:

A fragrance machine includes:
a housing, wherein the housing is provided with a hollow part in the middle;
an atomizing assembly, wherein the atomizing assembly includes a connecting pipe, a liquid storage bottle, an air pump, an atomizing pipe, a mounting block, an inserting pipe and an air nozzle; one end of the connecting pipe is closed and limited in the housing; an opened end of the connecting pipe is communicated with the atmosphere outside the housing; the liquid storage bottle is limited in the hollow part of the housing; an opened end of the liquid storage bottle is detachably sealed and fixed with a closed end of the connecting pipe after passing through a housing wall of the housing; a closed end of the connecting pipe is provided with connecting holes and a blind mounting hole, which are all limited at an inner side of the opened end of the liquid storage bottle; an air inlet channel is integrally formed in the connecting pipe; the air pump is limited in the housing; both ends of the air inlet channel are respectively communicated with the blind mounting hole and an air outlet end of the air pump; one end of the atomizing pipe is embedded in the blind mounting hole, and the mounting block is integrally formed at the other end; one side of the mounting block coincides with an axis of the atomizing pipe and is provided with a liquid spray hole; the inserting pipe is integrally formed on the mounting block, is limited in the liquid storage bottle and is communicated with the liquid spray hole; a tubular air nozzle is tightly embedded in the atomizing pipe; the air nozzle is communicated with the air inlet channel, and has an air spray direction perpendicular to a liquid spray direction of the liquid spray hole; and an axis line of the air nozzle intersects that of the liquid spray hole;
a power supply, wherein the power supply mounted in the housing and electrically connected with the air pump.

According to the above technical solution, compared with the prior art, the present invention discloses a fragrance machine; the housing of the fragrance machine in the present invention is provided with the hollow part; and the liquid storage bottle is detachably mounted at the hollow part of the housing, so that an operator can conveniently disassemble and replace the liquid storage bottle, the liquid level of fragrance liquid in the liquid storage bottle is directly visible to a user, and the user can supplement the fragrance liquid in time. The atomizing assembly in the present invention has simple structure and good atomization effect.

Preferably, the fragrance machine further includes a limiting pipe, a rifled pipe, a first cover plate, a mist outlet pipe and a second cover plate. A housing wall of the housing is provided with a mounting hole and a through hole which are communicated with the interior of the housing; the limiting pipe is integrally formed on an inner wall of the housing; and the through hole is coaxially limited at the inner side of the limiting pipe. The closed end of the connecting pipe is embedded in the limiting pipe; the rifled pipe is coaxially and integrally formed at the outer side of the closed end of the connecting pipe; the connecting holes and the blind mounting hole are all limited at the inner side of the rifled pipe; and an opened end of the liquid storage bottle is screwed with the rifled pipe after passing through the through hole. The opened end of the connecting pipe is blocked with the first cover plate; a plate surface of one side of the first cover plate far away from the connecting pipe is tightly abutted against the inner wall of the housing; and the first cover plate is perforated and vertically inserted with the mist outlet pipe. The second cover plate is tightly embedded in the mounting hole; the second cover plate is provided with a pocket hole; and the mist outlet pipe is tightly inserted into the pocket hole. The connecting pipe can be reliably limited in the housing; the liquid storage bottle can be reliably and detachably connected with the connecting pipe; and the air mixed with atomized fragrance liquid can be reliably discharged from the mist outlet pipe.

Preferably, the fragrance machine further includes a charging interface and a switch. The power supply is a rechargeable battery; the housing is perforated and is provided with the charging interface and the switch; the charging interface is electrically connected with the power supply; and the switch is electrically connected between the power supply and the air pump in series. The fragrance machine can be carried at any time without external power supply; and the air pump can be controlled by the switch.

Preferably, the atomizing assembly further includes a hose; one end of the hose is connected with the inserting pipe; and the other end of the hose can be abutted against an inner bottle bottom of the liquid storage bottle. The inserting pipe can suck the fragrance liquid at the bottle bottom of the liquid storage bottle through the hose.

Preferably, the atomizing assembly further includes a plug-in pipe and an air guide pipe. The plug-in pipe is fixed on an outer wall of the connecting pipe; one end of the plug-in pipe is communicated with the air inlet channel; and both ends of the air guide pipe are respectively communicated with the air outlet end of the air pump and the plug-in pipe. The air pump can be reliably communicated with the air inlet channel.

Preferably, a side of the mounting block, which coincides with the axis of the atomizing pipe, is provided with a splayed open slot. A small opening end of the open slot points to the inserting pipe; a diversion block is integrally formed in the center of a slot bottom wall of the open slot; and the liquid spray hole is formed in the diversion block. The above design makes the spray direction of the spray hole more concentrated, which contributes to improving the atomization effect.

### BRIEF DESCRIPTION OF DRAWINGS

To more clearly describe the technical solutions in the embodiments of the present invention or in the prior art, the drawings required to be used in the description of the embodiments or the prior art will be simply presented below. Apparently, the drawings in the following description are merely embodiments of the present invention, and for those ordinary skilled in the art, other drawings can also be obtained according to the drawings without contributing creative labor.

FIG. 1 is an overall axonometric view of a fragrance machine;

FIG. 2 is a partial axonometric view 1 of a fragrance machine;

FIG. 3 is a partial axonometric view 2 of a fragrance machine;

FIG. 4 is a partial axonometric view 3 of a fragrance machine;

FIG. 5 is a partial axonometric view 4 of a fragrance machine; and

FIG. 6 is a partial axonometric view 5 of a fragrance machine.

In the figures:
01 housing; 010 front cover; 011 rear housing; 012 hollow part; 02 connecting pipe; 020 connecting hole; 021 blind mounting hole; 022 air inlet channel; 03 liquid storage bottle; 04 air pump; 05 atomizing pipe; 06 mounting block; 060 liquid spray hole; 061 open slot; 062 diversion block; 07 inserting pipe; 08 air nozzle; 09 hose; 10 plug-in pipe; 11 air guide pipe; 12 power supply; 13 limiting pipe; 14 rifled pipe; 15 first cover plate; 16 mist outlet pipe; 17 second cover plate; 18 charging interface; and 19 switch.

### DETAILED DESCRIPTION OF THE EMBODIMENTS

The technical solutions in embodiments of the present invention will be clearly and fully described below. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

The present invention discloses a fragrance machine. A housing 01 of the fragrance machine in the present invention is provided with a hollow part, and a liquid storage bottle 03 is detachably mounted at the hollow part of the housing 01, so that an operator can conveniently disassemble and replace the liquid storage bottle 03, the liquid level of fragrance liquid in the liquid storage bottle 03 is directly visible to a user, and the user can supplement the fragrance liquid in time. An atomizing assembly in the present invention has good atomization effect. A splayed open slot 061 and a diversion block 062 are arranged on the mounting block 06; and a liquid spray hole 060 is formed in the diversion block 062. Through the arrangement, the air spray effect of an air nozzle 08 can be improved, thereby ensuring that the high-speed air sprayed from the air nozzle 08 can thoroughly blow the fragrance liquid sprayed from the liquid spray hole 060 into extremely fine liquid particles, and greatly improving the atomization effect of the atomizing assembly. The air nozzle 08 and the liquid spray hole 060 are both intensively arranged on the atomizing pipe 05. The arrangement can ensure that relative positions of the air nozzle 08 and the liquid spray hole 060 will not change, thereby ensuring the stability of the atomization effect of the atomizing assembly.

### Embodiment

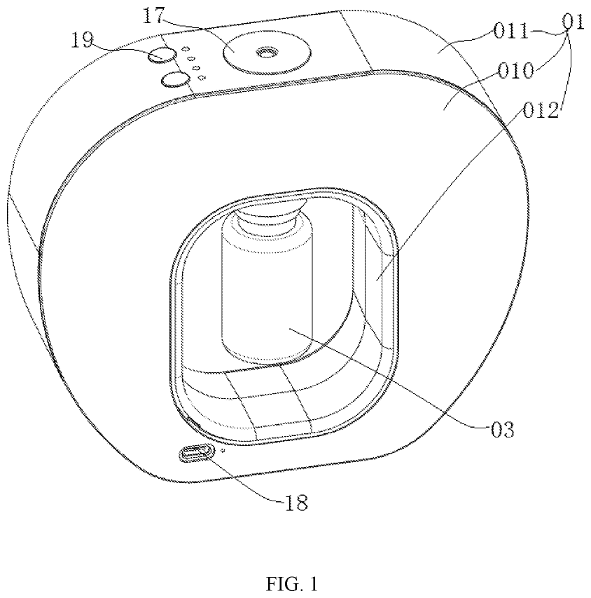

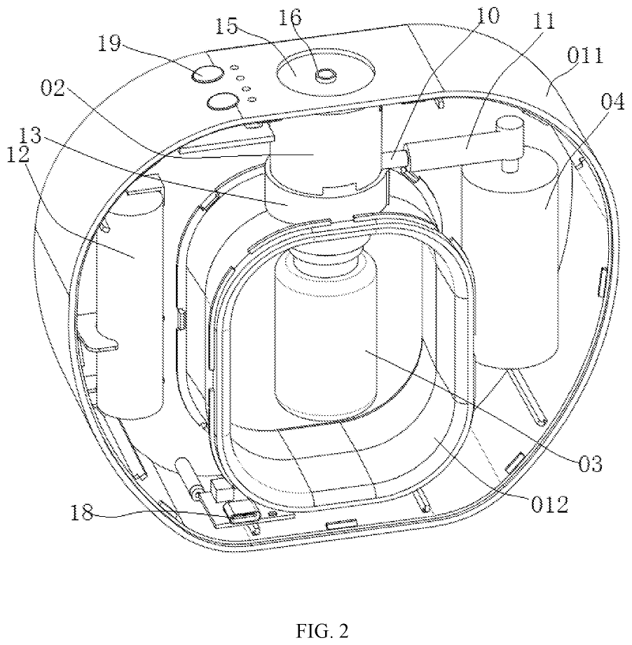

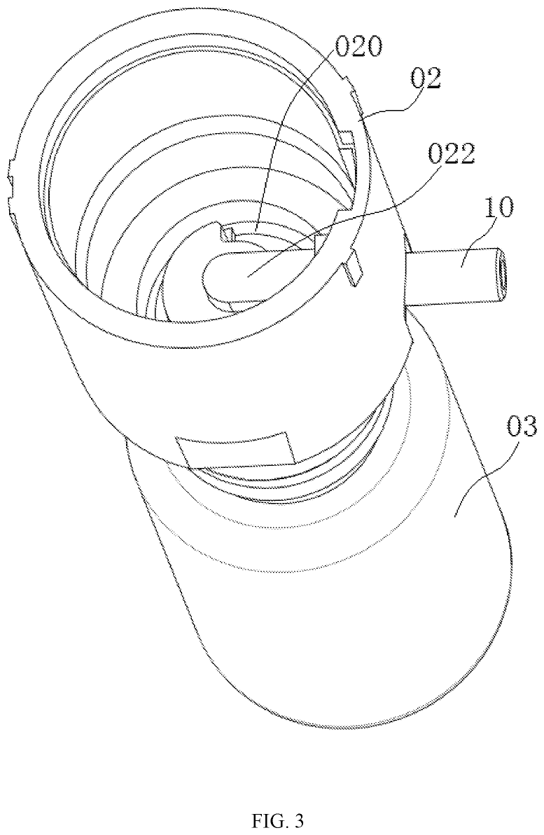

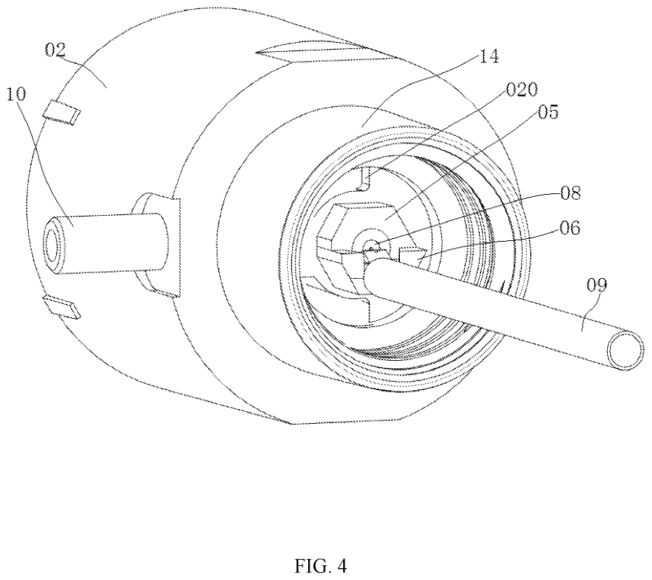

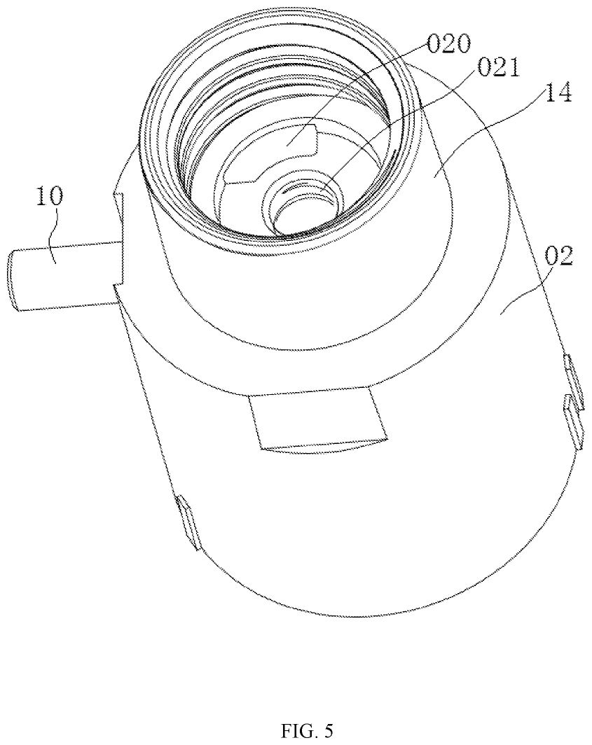

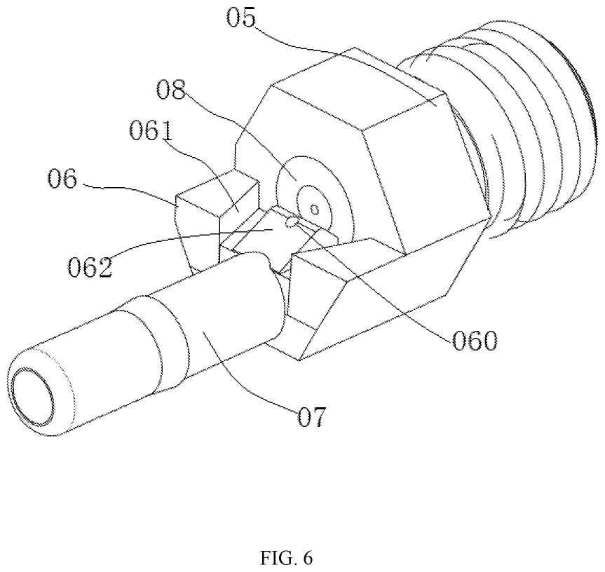

Referring to FIGS. 1-6, which are overall and partial structural schematic diagrams of an embodiment of the present invention, the present invention specifically discloses a fragrance machine, including:

a housing 01, an atomizing assembly and a power supply 12, wherein the housing 01 needs to be placed horizontally; a cross-sectional outline of the housing 01 is apple-shaped; the housing 01 is provided with a hollow part with a rectangular cross section in the middle; and the housing 01 is composed of three parts, i.e., a front cover 010, a rear cover 011 and the hollow part 012; and the front cover 010, the rear cover 011 and the hollow part 012 are respectively connected together in a clamping manner.

The atomizing assembly includes a connecting pipe 02, a liquid storage bottle 03, an air pump 04, an atomizing pipe 05, a mounting block 06, an inserting pipe 07 and an air nozzle 08.

One end of the connecting pipe 02 is opened and the other end is closed; the connecting pipe 02 is limited in the housing 01; and an opened end of the connecting pipe 02 is communicated with the atmosphere outside the housing 01, i.e., atomized air flows out from the opened end of the connecting pipe 02. The liquid storage bottle 03 is limited in the hollow part of the housing 01. When the housing 01 is horizontally placed, the liquid storage bottle 03 is vertically arranged upwards; and the opened end of the liquid storage bottle 03 is detachably sealed and fixed with a closed end of the connecting pipe 02 after passing through a housing wall of the housing 01, so that the operator can directly remove the liquid storage bottle 03 from the hollow part of the housing 01 without opening the housing 01; and the arrangement is convenient for replacing the liquid storage bottle 03.

A blind mounting hole 021 is formed in the center of the closed end of the connecting pipe 02. Meanwhile, the closed end of the connecting pipe 02 is further provided with a plurality of connecting holes 020; the connecting holes 020 are arranged adjacent to the blind mounting hole 021; atomized fragrance liquid in the liquid storage bottle 03 needs to pass through the connecting holes 020 before being discharged out of the housing 01; and the connecting holes 020 and the blind mounting hole 021 are all limited at an inner side of the opened end of the liquid storage bottle 03. Namely, the connecting pipe 02 is communicated with the interior of the liquid storage bottle 03 through the connecting holes 020; an air inlet channel 022 is integrally formed in the connecting pipe 02; the air pump 04 is limited in the housing 01 and clamped and fixed with the rear housing 011 in the housing 01; both ends of the air inlet channel 022 are respectively communicated with the blind mounting hole 021 and the air outlet end of the air pump 04; and the air pump 04 pumps air into the blind mounting hole 021 through the air inlet channel 022.

One end of the atomizing pipe 05 is embedded or screwed in the blind mounting hole 021; and the mounting block 06 is integrally formed at the other end. One side of the mounting block 06 coincides with an axis of the atomizing pipe 05 and is provided with a liquid spray hole 060; an inserting pipe 07 is integrally formed on the mounting block 06; the inserting pipe 07 is limited in the liquid storage bottle 03 and communicated with the liquid spray hole 060; and the inserting pipe 07 can suck the fragrance liquid in the liquid storage bottle 03. A tubular air nozzle 08 is tightly embedded in the atomizing pipe 05; the air nozzle 08 is communicated with the air inlet channel 022 and has an air spray direction perpendicular to a liquid spray direction of the liquid spray hole 060; and an axis line of the air nozzle 08 intersects that of the liquid spray hole 060.

The power supply 12 is mounted in the housing 01, and clamped and fixed with the rear housing 011 of the housing 01; and the power supply 12 is rechargeable, and electrically connected with the air pump 04.

Further specifically, the fragrance machine further includes a limiting pipe 13, a rifled pipe 14, a first cover plate 15, a mist outlet pipe 16 and a second cover plate 17.

A housing wall of the housing 01 is provided with a mounting hole and a through hole which are communicated with the interior of the housing; the limiting pipe 13 is integrally formed on an inner wall of the housing 01; and the through hole is coaxially limited at the inner side of the limiting pipe 13. The mounting hole is formed in the rear housing 011 of the housing 01; and both the through hole and the limiting pipe 13 are arranged on the hollow part 012 of the housing 01.

The closed end of the connecting pipe 02 is embedded in the limiting pipe 13; the rifled pipe 14 is coaxially and integrally formed at the outer side of the closed end of the connecting pipe 02; an opened end of the liquid storage bottle 03 is provided with a male thread connection section; the connecting holes 020 and the blind mounting hole 021 are all limited at the inner side of the rifled pipe 14; and the opened end of the liquid storage bottle 03 is screwed with the rifled pipe 14 after passing through the through hole. The opened end of the connecting pipe 02 is blocked with a circular first cover plate 15; a plate surface of one side of the first cover plate 15 far away from the connecting pipe 02 is tightly abutted against the inner wall of the housing 01; and the first cover plate 15 is perforated in the center and vertically inserted with the mist outlet pipe 16. A circular second cover plate 17 is tightly embedded in a circular mounting hole; the second cover plate 17 is provided with a pocket hole; and the mist outlet pipe 16 is tightly inserted into the pocket hole. The atomized fragrance liquid in the liquid storage bottle 03 can be discharged into the atmosphere outside the housing 01 after passing through the mist outlet pipe 16.

Further specifically, the fragrance machine further includes a charging interface 18 and a switch 19. The power supply 12 is a rechargeable battery; the housing 01 is perforated and is provided with the charging interface 18 and the switch 19; the charging interface 18 is arranged on the front cover 010, and the charging interface 18 is electrically connected with the power supply 12; and the switch 19 is arranged on the rear housing 011, and the switch 19 is electrically connected between the power supply 12 and the air pump 04 in series.

The atomizing assembly further includes a hose 09; one end of the hose 09 is connected with the inserting pipe 07; and the other end of the hose 09 can be abutted against an inner bottle bottom of the liquid storage bottle 03. The length of the hose 09 is greater than or equal to that of the liquid storage bottle 03, so as to ensure that the inserting pipe 07 can suck the fragrance liquid 03 at the bottle bottom of the liquid storage bottle 03.

The atomizing assembly further includes a plug-in pipe 10 and an air guide pipe 11. The plug-in pipe 10 is fixed on an outer wall of the connecting pipe 02; one end of the plug-in pipe 10 is communicated with the air inlet channel 022; and both ends of the air guide pipe 11 are respectively communicated with the air outlet end of the air pump 04 and the plug-in pipe 10.

A side of the mounting block 06, which coincides with the axis of the atomizing pipe 05, is provided with a splayed open slot 061. A small opening end of the open slot 061 points to the inserting pipe 07; a diversion block 062 is integrally formed in the center of a slot bottom wall of the open slot 061; and the liquid spray hole 060 is formed in the diversion block 062. When the air nozzle 08 sprays air out, the flow velocity of the air is accelerated when passing through the diversion block 062 and the open slot 061, so that the air spray direction of the spray hole is more concentrated, and the liquid sprayed from the liquid spray hole 060 can be blown into extremely fine liquid particles, thereby greatly improving the atomization effect.

When the fragrance machine is in operation, the operator horizontally arranges the housing 01 and turns on the switch 19; and then, the air pump 04 starts pumping air into the liquid storage bottle 03. The air is sprayed out from the air nozzle 08 at high speed, to form a negative pressure near the air nozzle 08. The high-speed air sprayed out from the air nozzle 08 will be converged by the diversion block 062 and the open slot 061, so that a strong negative pressure will be generated near the liquid spray hole 060. The liquid spray hole 060 sucks the fragrance liquid in the liquid storage bottle 03 through the inserting pipe 07 and the hose 09. While the air nozzle 08 continuously sprays air out, the liquid spray hole 060 will also spray liquid out due to the action of the negative pressure; and the liquid sprayed out from the liquid spray hole 060 will be rapidly atomized into tiny droplets and be scattered. Because the air nozzle 08 blows air into the liquid storage bottle 03, a positive pressure will be formed in the liquid storage bottle 03; and the air mixed with the atomized fragrance liquid will be discharged from the mist outlet pipe 16.

Each embodiment in the description is described in a progressive way. The difference of each embodiment from each other is the focus of explanation. The same and similar parts among all of the embodiments can be referred to each other.

The above description of the disclosed embodiments enables those skilled in the art to realize or use the present invention. Many modifications to these embodiments will be apparent to those skilled in the art. The general principle defined herein can be realized in other embodiments without departing from the spirit or scope of the present invention. Therefore, the present invention will not be limited to these embodiments shown herein, but will conform to the widest scope consistent with the principle and novel features disclosed herein.

What is claimed is:

1. A fragrance machine, comprising:
a housing (01), wherein the housing (01) is provided with a hollow part in a middle;
an atomizing assembly, wherein the atomizing assembly comprises a connecting pipe (02), a liquid storage bottle (03), an air pump (04), an atomizing pipe (05), a mounting block (06), an inserting pipe (07) and an air nozzle (08); one end of the connecting pipe (02) is closed and limited in the housing (01); an opened end of the connecting pipe (02) is communicated with atmosphere outside the housing (01); the liquid storage bottle (03) is limited in the hollow part of the housing (01); an opened end of the liquid storage bottle (03) is detachably sealed and fixed with a closed end of the connecting pipe (02) after passing through a housing wall of the housing (01); a closed end of the connecting pipe (02) is provided with connecting holes (020) and a blind mounting hole (021); the connecting holes (020) and the blind mounting hole (021) are all limited at an inner side of the opened end of the liquid storage bottle (03); an air inlet channel (022) is integrally formed in the connecting pipe (02); the air pump (04) is limited in the housing (01); both ends of the air inlet channel (022) are respectively communicated with the blind mounting hole (021) and an air outlet end of the air pump (04); one end of the atomizing pipe (05) is embedded in the blind mounting hole (021), and the mounting block (06) is integrally formed at the other end; one side of the mounting block (06) coincides with an axis of the atomizing pipe (05) and is provided with a liquid spray hole (060); the inserting pipe (07) is integrally formed on the mounting block (06); the inserting pipe (07) is limited in the liquid storage bottle (03) and is communicated with the liquid spray hole (060); a tubular air nozzle (08) is tightly embedded in the atomizing pipe (05); the air nozzle (08) is communicated with the air inlet channel (022), and has an air spray direction perpendicular to a liquid spray direction of the liquid spray hole (060); and an axis line of the air nozzle (08) intersects an axis line of the liquid spray hole (060);
a power supply (12), wherein the power supply (12) is mounted in the housing (01) and electrically connected with the air pump (04).

2. The fragrance machine according to claim 1, further comprising a limiting pipe (13), a rifled pipe (14), a first cover plate (15), a mist outlet pipe (16) and a second cover plate (17), wherein a housing wall of the housing (01) is provided with a mounting hole and a through hole which are communicated with the interior of the housing; the limiting pipe (13) is integrally formed on an inner wall of the housing (01); the through hole is coaxially limited at the inner side of the limiting pipe (13); the closed end of the connecting pipe (02) is embedded in the limiting pipe (13); the rifled pipe (14) is coaxially and integrally formed at the outer side of the closed end of the connecting pipe (02); the connecting holes (020) and the blind mounting hole (021) are all limited at the inner side of the rifled pipe (14); an opened end of the liquid storage bottle (03) is screwed with the rifled pipe (14) after passing through the through hole; the opened end of the connecting pipe (02) is blocked with the first cover plate (15); a plate surface of one side of the first cover plate (15) far away from the connecting pipe (02) is tightly abutted against the inner wall of the housing (01); the first cover plate (15) is perforated and vertically inserted with the mist outlet pipe (16); the second cover plate (17) is tightly embedded in the mounting hole; the second cover plate (17) is provided with a pocket hole; and the mist outlet pipe (16) is tightly inserted into the pocket hole.

3. The fragrance machine according to claim 1, further comprising a charging interface (18) and a switch (19), wherein the power supply (12) is a rechargeable battery; the housing (01) is perforated and is provided with the charging interface (18) and the switch (19); the charging interface (18) is electrically connected with the power supply (12); and the switch (19) is electrically connected between the power supply (12) and the air pump (04) in series.

4. The fragrance machine according to claim 1, wherein the atomizing assembly further comprises a hose (09); one end of the hose (09) is connected with the inserting pipe (07); and the other end of the hose (09) is capable of being abutted against an inner bottle bottom of the liquid storage bottle (03).

5. The fragrance machine according to claim 1, wherein the atomizing assembly further comprises a plug-in pipe (10) and an air guide pipe (11); the plug-in pipe (10) is fixed on an outer wall of the connecting pipe (02); one end of the plug-in pipe (10) is communicated with the air inlet channel (022); and both ends of the air guide pipe (11) are respectively communicated with the air outlet end of the air pump (04) and the plug-in pipe (10).

6. The fragrance machine according to claim 1, wherein a side of the mounting block (06), which coincides with the axis of the atomizing pipe (05), is provided with a splayed open slot (061); a small opening end of the open slot (061) points to the inserting pipe (07); a diversion block (062) is integrally formed in the center of a slot bottom wall of the open slot (061); and the liquid spray hole (060) is formed in the diversion block (062).
